(19) United States
(12) Patent Application Publication
(10) Pub. No.: US 2012/0259376 A1
(43) Pub. Date: Oct. 11, 2012

(54) Charge Storage System

(75) Inventor: Glenn Godden, Edmonds, WA (US)

(73) Assignee: Empire Technology Development LLC

(21) Appl. No.: 13/392,026

(22) PCT Filed: Apr. 8, 2011
(86) PCT No.: PCT/US11/31783

§ 371 Date: Feb. 23, 2012

(51) Int. Cl. A61N 1/36; H02J 7/00; H01G 4/00; H05K 3/00

(52) U.S. Cl. 607/2; 29/829; 320/166; 361/301.1

(57) ABSTRACT

An apparatus includes a charge storage device. The charge storage device includes a first plate, a second plate, and a dielectric. The dielectric separates the first and the second plate. The first plate is configured to hold a first charge, and the second plate is configured to hold a second, opposite, charge. The charge storage device is housed in a product receptacle that is configured to charge the first plate and the second plate such that the charge storage device is able to generate an electrical current.

### BACKGROUND

The following description is provided to assist the understanding of the reader. None of the information provided or references cited is admitted to be prior art.

A capacitor, which is a device that can store an electrical charge, may include a pair of conductors that are separated by a dielectric material. An electrical charge can be stored by applying a voltage across the pair of conductors, which creates a static electric field in the dielectric material. The stored electrical charge remains after the being disconnected from the charging voltage. A charged capacitor can, therefore, be used similar to a battery.

### SUMMARY

An illustrative apparatus includes a charge storage device that is composed of a first plate and a second plate. The first plate can hold a first charge and the second plate can hold a second, opposite charge. The charge storage device also includes a dielectric that separates the first and second plates. The apparatus also includes a product receptacle that can house the charge storage device. In addition, the product receptacle can charge the first and second plate when the product receptacle is opened, which allows the charge storage device to generate an electrical current.

In another embodiment, the charge storage device further includes a first electrode mounted to the first plate and a second electrode mounted to the second plate. In another embodiment, the product receptacle includes a battery connected to the first electrode and the second electrode, where the battery can provide the first charge to the first electrode and the second charge to the second electrode upon opening of the product receptacle. In yet another embodiment, opening of the product receptacle charges the first plate with the first charge and the second plate with the second charge through triboclectric charging.

In another embodiment, the first plate and the second plate are composed of at least one of copper, carbon, manganese dioxide, or iron. In one embodiment, the dielectric is composed of at least one of wax, sugar, agar, or sea weed.

In yet another embodiment, a food product can be housed in the product receptacle and the food product can include the charge storage device. In a related embodiment, the charge storage device is composed of food-grade components. In some embodiments, the food product can include at least one of a gum, a candy, or a cracker.

In another embodiment, a vitamin can be housed in the product receptacle. In a related embodiment, the charge storage device is incorporated in or on the vitamin. In another related embodiment, the vitamin can be incorporated in or on at least one of the first plate, the second plate, and the dielectric.

In yet another embodiment, the apparatus can include a resistor that is in electrical communication with at least one of the first plate and the second plate. In one embodiment, the resistor can be composed of carbon.

In another embodiment, placement of the charge storage device in contact with a subject releases the electrical current, and the electrical current is configured to increase production of a biological fluid. In one embodiment, the biological fluid can be saliva.

In yet another embodiment, a non-conductive layer that can encapsulate the charge storage device. In one embodiment, the non-conductive layer is waterproof. In some embodiments, the non-conductive layer includes at least one of wax, sucrose, or chocolate.

In another embodiment, the apparatus includes a pill housed in the product receptacle and the charge storage device can be incorporated in or on the pill. In one embodiment, the pill can be configured to generate the electrical current in stomach tissue of a subject.

In yet another embodiment, the apparatus includes a dissolvable strip housed in the producfreceptacle and the charge storage device can be incorporated in or on the dissolvable strip. In some embodiments, the dissolvable strip can include a breath freshener, a teeth whitener, or a drug. In other embodiments, the electrical current can be configured to reduce growth of flora within a mouth, combat gum disease, or enhance absorption of the drug in a subject.

In another embodiment, the apparatus includes a patch housed in the product receptacle and the charge storage device is incorporated in or on the patch. In one embodiment, at least a portion of the first plate and at least a portion of the second plate can contact skin of a subject to deliver the electrical current to the subject. In another embodiment, at least the portion of the first plate includes a first electrode, and at least the portion of the second plate includes a second electrode. In one embodiment, the charge storage device includes one or more anti-bacterial agents. In another embodiment, the patch can be used to treat acne by delivering the electrical current to a subject. In some embodiments, the electrical current can stimulate a muscle or prevent secretion of a bodily fluid. In one embodiment, the bodily fluid includes sweat. In other embodiments, the patch can include a fragrance or a drug. In one embodiment, the electrical current is configured to enhance absorption of the drug.

In yet another embodiment, the patch can be a foot patch, and the electrical current can reduce production of sweat from a foot of a subject. In another embodiment, the patch can include a cleaning patch that includes a cleaning agent, and the electrical current is released upon placement of the patch on a surface that is to be cleaned. The electrical current can enhance performance of the cleaning agent. In another embodiment, the electrical current can be used to provide sexual stimulation. In one embodiment, the apparatus includes a dissolvable strip housed in the product receptacle, and the charge storage device is incorporated in or on the dissolvable strip. The dissolvable strip is configured such that it can be inserted into a vagina.

In an illustrative process, a charge storage device is generated by printing a dielectric onto a medium, printing a first plate onto a first side of the dielectric, and printing a second plate onto a second side of the dielectric such that the dielectric separates the first plate from the second plate. The first plate can hold a first charge and the second plate can hold a second, opposite charge. The charge storage device can be incorporated into a product receptacle that is configured to charge the first plate and the second plate upon opening such that the charge storage device is able to generate an electrical current.

In one embodiment, the medium includes rice paper. In one embodiment, the charge storage device can be incorporated into a food product that is housed in the product receptacle. In some embodiments, the food product can include at least one of a gum, a candy, or a cracker.

In some embodiments, printing operations arc performed by a laser jet printer or a printing press. In another embodiment, a wax printer can be used to print at least the dielectric. In one embodiment, the dielectric includes at least one of wax, sugar, agar, or sea weed. In one embodiment, the first plate and the second plate are made of at least one of copper, carbon, manganese dioxide, or iron.

Another embodiment includes encapsulating the charge storage device with a non-conductive layer. In one embodiment, the non-conductive layer is waterproof. The non-conductive layer can be at least one of wax, sucrose, or chocolate.

In another embodiment, a resistor is printed so that the resistor is electrically connected to at least one of the first plate or the second plate. In one embodiment, the resistor can be composed of carbon.

In another embodiment, a battery is incorporated into the product receptacle. In one embodiment, the battery can provide the first charge to the first plate and the second charge to the second plate upon opening of the product receptacle.

Another embodiment includes providing, upon opening of the product receptacle, the first charge to the first plate and the second charge to the second plate via triboelectric charging.

In yet another embodiment, the electrical current can increase production of saliva, and the charge storage device can be administered to treat dry mouth. In another embodiment, the charge storage device can be incorporated into at least one of a vitamin or a drug and the electrical current can enhance provision of the vitamin or the drug to a subject. In yet another embodiment, at least one of a vitamin or a drug can be incorporated in or on at least one of the first plate, the second plate, or the dielectric.

In another embodiment, the charge storage device can be incorporated in or on a pill. In one embodiment, the pill can generate the electrical current in stomach tissue of a subject.

In yet another embodiment, the charge storage device can be incorporated in or on a dissolvable strip that is housed in the product receptacle. In some embodiments, the dissolvable strip can include at least one of a breath freshener, a teeth whitener, or a drug.

In another embodiment, the charge storage device can be incorporated in or on a patch. In one embodiment, at least a portion of the first plate and at least a portion of the second plate are configured to contact skin of a subject to deliver the electrical current to the subject. In some embodiments, the patch can be used to treat acne, stimulate a muscle, deliver a drug, or prevent secretion of a bodily fluid.

In yet another embodiment, the electrical current can provide sexual stimulation.

Another illustrative apparatus includes a charge storage device that includes a first plate and a second plate. The first plate can hold a first charge and the second plate can hold a second, opposite charge. The charge storage device also can include a dielectric that separates the first plate from the second plate. An edible product can be configured to house the charge storage device. In one embodiment, a product receptacle can house the edible product.

In one embodiment, the product receptacle is configured to charge the first plate and the second plate upon opening of the product receptacle such that the charge storage device is able to generate an electrical current. In another embodiment, the first charge can be applied to the first plate and the second charge can be applied to the second plate prior to placement of the edible product into the product receptacle.

Another illustrative process includes generating a charge storage device by printing a dielectric onto a medium, printing a first plate onto a first side of the dielectric, and printing a second plate onto a second side of the dielectric such that the dielectric separates the first plate from the second plate. The first plate can hold a first charge, and the second plate can hold a second, opposite charge. The process also includes incorporating the charge storage device into an edible product.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a charge storage device in accordance with an illustrative embodiment.

FIG. 2 illustrates a charging circuit that can charge a charge storage device in accordance with an illustrative embodiment.

FIG. 3 illustrates a circuit including a charge storage device in accordance with an illustrative embodiment.

FIG. 4 illustrates a charge storage device incorporated into a product in accordance with an illustrative embodiment.

FIG. 5 illustrates a product receptacle including a battery in accordance with an illustrative embodiment.

FIG. 6 illustrates a product receptacle that utilizes triboelectric charging of a charge storage device in accordance with an illustrative embodiment.

FIG. 7 is a flow diagram depicting operations performed in generating a charge storage device in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

Described herein are illustrative methods and apparatuses relating to a charge storage device that can be configured to deliver an electrical current/voltage. Non-limiting uses of the charge storage device may include incorporating the charge storage device into an edible product, using the charge storage device to enhance a cleaning agent, using the charge storage device topically on humans, animals, plants, etc. to treat a condition, to stimulate a muscle, to reduce sweat, to provide sexual pleasure, to treat dry mouth or canker sores, to deliver a vitamin or drug, etc. These and other illustrative embodiments are described in greater detail below. It will be understood that the above embodiments and configurations are given as illustrative examples only and that other configurations of the charge storage device will be apparent to those of skill in the art in light of the present disclosure. Additional details and embodiments are described with reference to the figures.

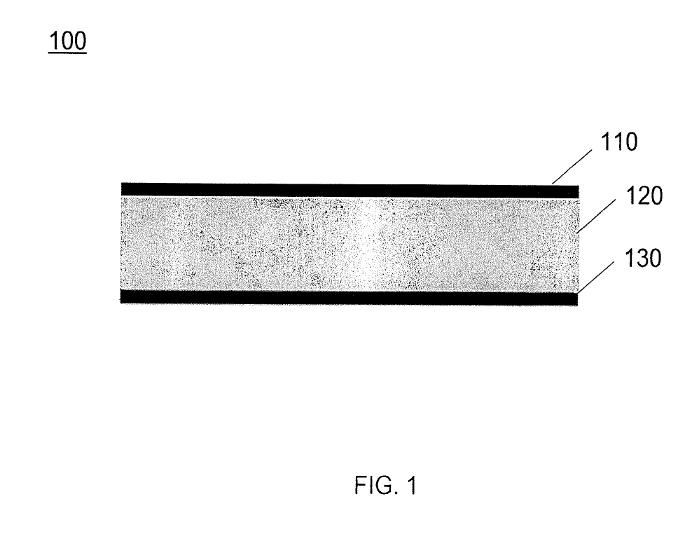

FIG. 1 illustrates a charge storage device 100 in accordance with an illustrative embodiment. The charge storage device 100 includes a first plate 110, a second plate 130, and a dielectric 120 that separates the first plate 110 and the second plate 130. In one embodiment, the first plate 110, second plate 130, and the dielectric 120 may be made of food-grade products. For example, food-grade carbon, copper, nickel, iron, manganese dioxide, phosphorus, iodine, magnesium, selenium, manganese; chromium, molybdenum chloride, etc. arc non-limiting examples of materials that can be used to form the first plate 110 and/or the second plate 130. The dielectric 120 can be made up of materials including, but not limited to, wax, sugar, agar agar, sea weed, algae, chocolate, dried oils (e.g., olive oil), lecithin, gelatin, pectin, calcium, etc. As discussed in further detail below, a charge storage device 100 including food-grade products may be incorporated into various types of food products, such as, but not limited to, snack foods, prepared foods, candies, etc. Specific examples of food products that the charge storage device 100 may be incorporated into include, but are not limited to, crackers, cookies, jam, dried fruits, gum, chocolate, caramel, bread, etc.

In one embodiment, the first plate 110, the second plate 130, and/or the dielectric 120 can be composed at least in part by vitamins and/or minerals. As such, upon ingestion of the charge storage device 100 by a subject, the charge storage device 100 may deliver the vitamins and/or minerals to the subject. For example, the first plate 110, the second plate 130, and/or the dielectric 120 may include materials such as, but not limited to, copper, zinc, nickel, or iron. In one embodiment, the amount of vitamins and/or minerals used is around 0.05 of the recommended daily intake of the used vitamins and/or minerals.

In the illustrated embodiment of FIG. 1, the dielectric 120 separates the first plate 110 and the second plate 130 such that the charge storage device 100 functions as a capacitor. In an illustrative embodiment, the charge storage device 100 can be charged by applying a voltage differential to the first plate 110 and the second plate 130 across the dielectric 120. Any voltage generating or storage device known to those of skill in the art may be used to charge the charge storage device 100. In one embodiment, the first plate 110 and the second plate 130 may be devoid of any electrical charge prior to the application of the voltage. In an alternative embodiment, the voltage may be used to recharge a partially charged charge storage device 100.

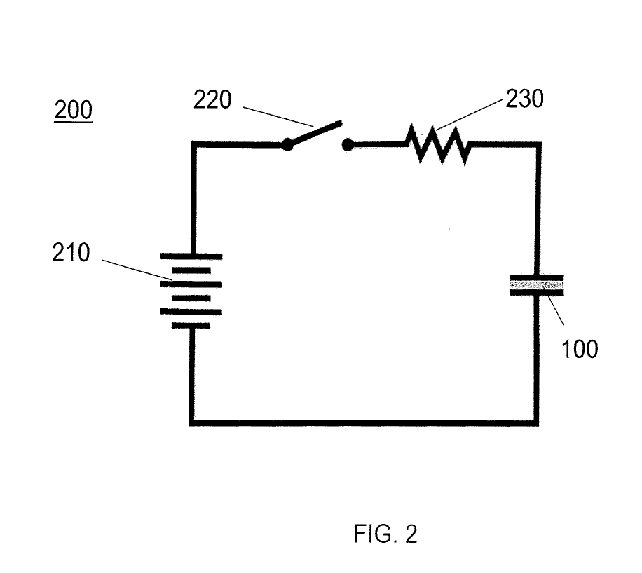

FIG. 2 illustrates a charging circuit 200 that can charge a charge storage device in accordance with an illustrative embodiment. The charging circuit 200 includes a voltage source 210, a resistor 230, the charge storage device 100, and a switch 220. Closing the switch 220 causes a voltage difference to be applied between the first plate 110 and the second plate 130, and across the dielectric 120. When the charging circuit 200 is operating, a first charge is applied to the first plate 110 and a second, opposite charge is applied to the second plate 130. Based upon Kirchoff's voltage law, the voltage of the charge storage device 100 will, in theory, be equal to the voltage of the voltage source 210. In practice, the voltage across the charge storage device 100 may be slightly less than the voltage across the voltage source 210 due to internal circuit resistance/losses as known to those of skill in the art.

Once the charge storage device 100 is charged, the charge storage device 100 can be disconnected from the voltage source 210 and removed from the charging circuit 200. The charge storage device 100 will retain the stored charge, subject to dielectric losses over time, and can act as a voltage source for a circuit to which the charge storage device 100 is connected. Once connected to a circuit, the charge storage device 100 can generate an electrical current that flows through the circuit.

In one illustrative embodiment, the charge storage device 100 can be charged prior to being packaged or incorporated into a product. In this embodiment, a voltage source, such as voltage source 210 in the charging circuit 200, can be connected to the first plate 110 and the second plate 130. The amount of time that it takes for the voltage source to fully charge the charge storage device 100 can be based at least in part upon the value of resistor 230 and the capacitance of the charge storage device 100, as known to those of skill in the art. Once connected to the charging circuit 200, the voltage, V_{c}, of the charge storage device 100 can be calculated based upon the following the formula:

$${V}_{c} = {V}_{S} ( 1 - {e}^{\frac{- t}{RC}} ) ,$$

where Vₛ is the voltage of the voltage source 210, R is the value of resistor 230, t is the amount of time the charging circuit 200 has been active, and C is the capacitance of the charge storage device 100. After the charge storage device 100 has been connected to a voltage source for a sufficient amount of time, the charge storage device 100 will have a charge that is approximately equivalent to that of the voltage source 210. In one embodiment, the resistor 230 is not used in the charging circuit, which reduces the amount of time that it takes to fully charge the charge storage device 100.

In another embodiment, described in further detail below, the charge storage device 100 can be placed, uncharged, in a product receptacle that has the ability to charge the charge storage device 100. The charge storage device 100 can be charged at the point of sale or at a later time prior to the usage of the charge storage device 100. In another embodiment, the charge storage device 100 can be placed, after being charged, in a product receptacle that has the ability to recharge the charge storage device 100 and/or maintain the charge on the charge storage device 100. A charge storage device 100 that uses a quality dielectric and has no or minimal leakage from the negatively charge plate to the positively charged plate can retain its charges for hundreds of days.

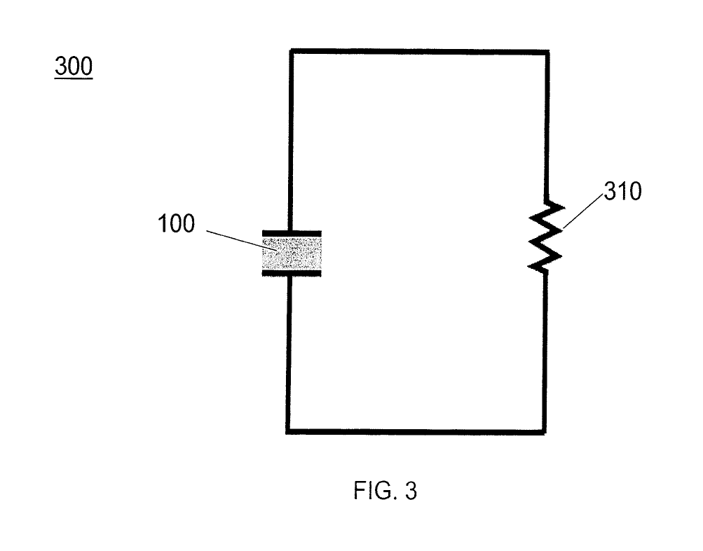

FIG. 3 illustrates a circuit 300 including a charge storage device 100 in accordance with an illustrative embodiment. The circuit 300 includes the charge storage device 100 and a resistor 310. If the charge storage device 100 has been charged, for example using the charging circuit 200 illustrated in FIG. 2, the charge storage device 100 can act as a battery and drive the circuit 300. In one embodiment, the charge storage device 100 can be incorporated into a food product such as, but not limited to, a gum or a candy. When placed into the mouth of a subject, various components of the subject's mouth can act as the resistor 310. For example, the resistor 310 can be formed from one or more parts of the mouth, such as, but not limited to, the tongue, teeth, gums, the walls of the mouth, and/or fluid associated with the mouth. As such, upon placement of the charge storage device 100 into a mouth, the circuit 300 can be formed and a current can flow through the circuit. In one embodiment, the current can be detectable by the subject (or user) as a tingling of the parts of the mouth that form the circuit 300. In another embodiment, the resistor 310 can be incorporated into a product along with the charge storage device 100. For example, as described in further detail below, the charge storage device 100 and the resistor 310 can be incorporated into a patch. When the patch comes into contact with a conducting surfacing, such as skin, the circuit 300 can be formed through the resistor 310 and the conducting surface to allow the charge storage device 100 to generate a current through the circuit 300, and thus through the conducting surface.

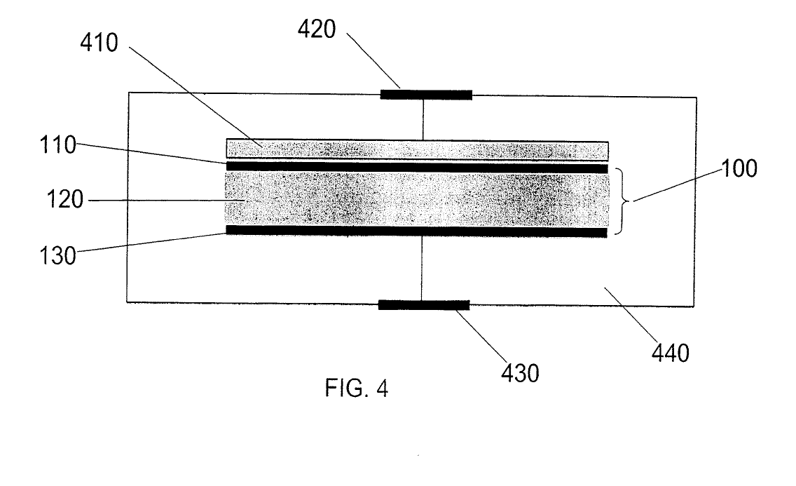

FIG. 4 illustrates a charge storage device 100 incorporated into a product 440 in accordance with an illustrative embodiment. The charge storage device 100 can be incorporated into various products such as, but not limited to, gum, cleaning sponges, snack foods, prepared foods, candies, breath strips, crackers, patches, etc. In an illustrative embodiment, the charge storage device 100 can be connected to a resistor 410 and to a first electrode 420 and a second electrode 430. The resistor 410 be made of materials including, but not limited to, carbon, salt, nickel chromium, etc. The resistor 410 can be connected to the first plate 110 or the second plate 130 of the charge storage device 100. In another embodiment, multiple resistors can be used that can be connected to the first plate 110 and/or the second plate 130. In other embodiments, the product may not include a resistor. The electrodes 420 and 430 can be used to connect the charge storage device 100 to a circuit, which can then be driven by the charge storage device 100. In an illustrative example, the electrodes 420 and 430 can be located on the surface of a piece of chewing gum, with the charge storage device 100 embedded within the piece of gum. When placed in a subject's mouth, the electrodes 420 and 430 can form a circuit with the subject's mouth, creating a circuit, such as the circuit 300 shown in FIG. 3. The charge storage device 100 can generate a current through the circuit. In an illustrative embodiment, the current can be used to stimulate the production of saliva in a subject's mouth, and therefore, the gum can be administered to treat or prevent dry mouth. The current may also be used as a flavor enhancement to improve the chewing experience of the gum. The charge storage device 100 can similarly be incorporated into other food products such as crackers, bread, dried fruit, chocolate, caramel, cookies, pitas, chips, etc.

In one embodiment, the electrodes 420 and 430 can be located on the same surface (or side) of a product 440. An illustrative example is a cleaning sponge or cleaning patch. The cleaning sponge or cleaning patch can include; but is not limited to, a cleaning agent, detergent, fragrance, etc. In such an embodiment, the electrodes 420 and 430 can both be located on the same surface of the cleaning sponge. When the cleaning sponge is placed on any conductive surface, a circuit formed with the electrodes 420 and 430 and the conductive surface can be driven by the charge storage device 100 to generate an electrical current. The electrical current generated in the circuit can be used to help loosen dirt and grease, and/or to enhance the cleaning ability of the cleaning agent or the detergent on the conductive surface.

In another embodiment, one or more charge storage devices 100 can be embedded in a patch or bandage that can be affixed to a subject with an adhesive or a strap. The patch can include the first plate 110, the second plate 130, and the dielectric 120, and can be attached to skin. Accordingly, the patch can be worn by the subject. In one embodiment, the first electrode 420 and the second electrode 430 can be located on one surface (or side) of the patch or bandage. When the patch or bandage is affixed, the surface of the patch that includes the electrodes 420 and 430 can be in contact with the subject such that the electrodes 420 and 430 are also in contact with the subject. The electrodes 420 and 430 can form a circuit with a portion of the subject's skin that can be driven by the charge storage device 100. The electrical current generated in the circuit can be used to effect delivery/absorption of a substance, such as, but not limited to, a drug. In one embodiment, the drug may be incorporated into one or more of the first plate 110, the dielectric 120, and the second plate 130. Alternatively, the drug may be incorporated into the material that forms the patch or bandage. In either embodiment, the generated electrical current can help a subject to absorb the drug. The electrical current can also be used to combat a virus such as the herpes virus, to stimulate muscles, etc. In addition, the current can also have an anti-bacterial effect and be used alone or in conjunction with delivery of a drug to combat acne. The current can also have an anti-viral effect and can be used to in the treatment of sores of the mouth, such as, but not limited to, canker sores. In another illustrative embodiment, the patch can include anti-bacterial and/or anti-viral agents. In another embodiment, the current from a charge storage device 100 can be used to treat various skin conditions. In one illustrative embodiment, a patch that incorporates one or more charge storage devices can be applied to a rash, an area of dry skin, and/or an area of irritated skin. The one or more charge storage devices can generate a current that flows through portions of the skin, which can be used to diminish itching in skin by providing a competing stimulus.

In another illustrative embodiment, a patch that incorporates one or more charge storage device's 100 can be used to treat wounds or burns. The patch can be applied to an affected portion of a subject's skin such that the charge storage device 100 can provide a current through the subject's skin tissue. The current can be used to affect the healing of the wound through, but not limited to, increasing blood flow, enhancing tissue oxygenation, preventing an infection, stimulating epidermal cell reproduction, etc. In some embodiments, the electrical current can reduce the amount of scar tissue of a healed wound, resulting in a smoother and thinner scar.

In another embodiment, one or more charge storage devices 100 contained in a patch can be used as an antiperspirant and/or deodorant. For example, the first plate 110 can include an aluminum complex, the second plate 130 can include carbon, and the dielectric 120 can be wax. Illustrative aluminum complexes include, but are not limited to, aluminum chloride, aluminum chlorohydrate, and aluminum-zirconium compounds. The electrodes 420 and 430 can form a circuit with a portion of the subject's skin, e.g., underarm, feet, etc. The current produced by the charge storage device 100 through a subject's skin can aid in the iontophoretic delivery of the aluminum-based complexes. Although not intended to be limited by theory, the aluminum-based complexes may aid in the formation of plugs in sweat glands, and thus, can help prevent perspiration. The aluminum-based complexes may also interact with keratin fibrils in sweat ducts and form a physical plug that prevents sweat from reaching the surface of skin. Accordingly, a patch can be used to combat excessive sweating in an area covered by the patch. The patch can also include fragrant materials. Accordingly, the patch can be used as a deodorant.

In another embodiment, one or more charge storage devices 100 may be attached to or printed on a pad such as a foot pad. Unlike the patch/bandage, the pad may not affixed to a subject. Rather, the pad can be placed into contact with the subject without the use of an adhesive, strap, etc. The pad can include the first plate 110, the second plate 130, and the dielectric 120, and can be placed in contact with skin. In one embodiment, the electrodes 420 and 430 can be in contact with a subject to form a circuit. In an illustrative embodiment, the pad can be worn in a shoe. The electrodes 420 and 430 can be in contact with the subject's foot and can cause an electrical current to flow through the subject's foot. The current that flows through the foot can have an anti-bacterial effect and can also decrease sweating of the foot. In one embodiment, the pad and/or charge storage device(s) may also include one or more antibacterial agents that work in conjunction with the electrical current. The pad and/or charge storage device(s) may also include a fragrance. Accordingly, in an illustrative embodiment, a foot pad that includes one or more charge storage devices 100 can be used to combat foot odors.

In another illustrative embodiment, a dissolvable strip can include one or more charge storage devices 100. The strip and/or charge storage devices 100 can include other components such that the strip can be used as a breath freshener, as a teeth whitener, to deliver medicine such as antacid medicine, cold medicine, nicotine, or anti-gas medicine, as an energy supplement, etc. In each of these embodiments, a charge storage device 100 can provide an electrical current that enhances the performance of the strip for its intended purpose. For instance, the electrical current may facilitate the delivery of a drug contained within the strip or a drug that is ingested simultaneously with the strip. In another embodiment, the current from the charge storage device(s) 100 may impede the flora of the mouth, and therefore, help protect against cavities and/or gum disease. In addition, the charge storage device 100 can increase the production and secretion of saliva, which also helps protect against cavities and/or gum disease.

In another embodiment, one or more charge storage devices 100 can be incorporated into a product that is used for sexual stimulation. In an illustrative embodiment, the one or more charge storage devices 100 can be attached to or printed on a thin dissolving film. Alternatively, the charge storage devices 100 can be incorporated into a patch, strip, pad, pill, etc. for providing sexual stimulation. In one embodiment, the electrodes 420 and 430 can be on one side of the film and can come into contact with a subject to form a circuit. The charge storage device(s) 100 can be inserted into any orifice such as a vagina, a mouth, etc. Alternatively, the charge storage device(s) 100 can be placed upon genitalia such as a penis. The one or more charge storage devices 100 can produce an electrical current that flows through the subject's orifices and/or genitals to provide sexual stimulation. In addition, the effects of the charge storage device 100 can be transferred from one person to another. For instance, current generated by a charge storage device 100 can be transferred between two individuals through kissing, fellatio, cunnilingus, sexual intercourse, etc. The electrical current can result in heightened sexual stimulation.

In another embodiment, the charge storage device 100 can be incorporated into a pill. A coating can surround the pill and encapsulate the anode chamber and the cathode chamber. The coating can be made of, but not limited to, e.g., gelatin, wax, hypromellose, methyl cellulose, hydroxypropyl cellulose, etc. In one embodiment, the pill can be swallowed by a subject and the coating can dissolve during digestion. Eventually, the charge storage device 100 can be exposed and can form a circuit using a subject's stomach and other digestive organs. In another embodiment, pressure applied to the pill can rupture the coating and expose the charge storage device 100. For example, the pressure can be applied by the pill being chewed, which can expose the charge storage device 100, such that a circuit can formed in a subject's mouth. In another embodiment, the pill or the coating can include a drug or vitamin, whose delivery is improved by the current generated by the charge storage device 100.

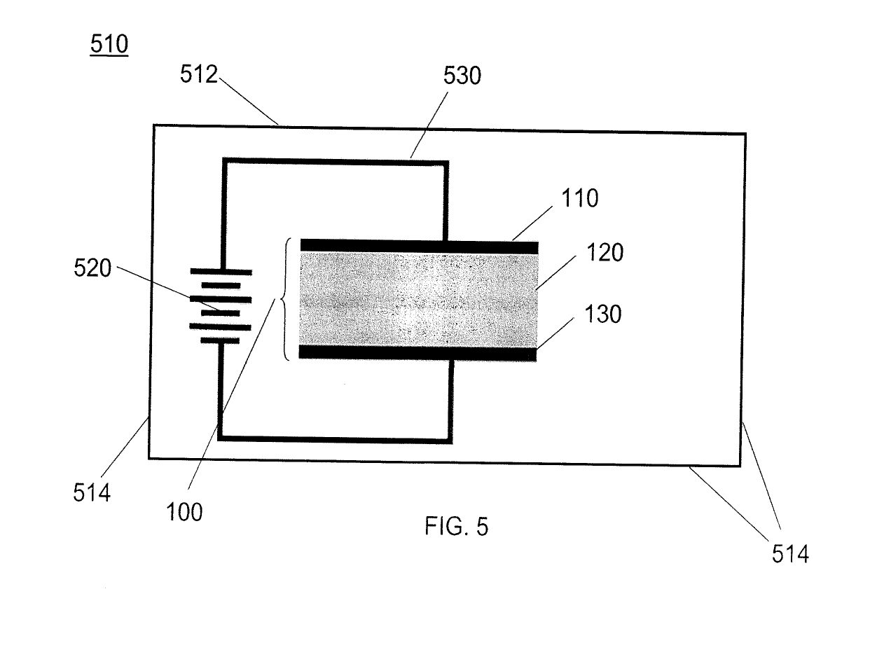

As described above, the charge storage device 100 can be charged before being placed in a packing receptacle. The charge storage device 100 can also be charged and/or recharged at some time after being placed in a packaging receptacle. FIG. 5 illustrates a product receptacle 510 including a battery 520 in accordance with an illustrative embodiment. In one embodiment, the battery 520 is a step up high voltage power supply. The battery 520 can drive a charging circuit 530 to charge the charge storage device 100. In one embodiment, the product receptacle 510 includes a top portion 512 and a base portion 514. The top portion 512 can be opened, for example by being peeled away from the base portion 514 of the product receptacle 510 to expose the charge storage device 100 and/or a product that includes the charge storage device 100. The top portion 512 can include a non-conductive separator that can be located between the battery 520 and the charging circuit 530. As such, the non-conductive separator can prevent the battery 520 from driving the charging circuit 530 until the product receptacle 510 is opened by, for example, peeling the top portion 512 from the base portion 514. When the top portion 512 is peeled away, the non-conductive separator can be moved such that the battery 520 electrically connects to the charging circuit 530 to charge or recharge the charge storage device 100. The product receptacle 510 can also be opened by, but not limited to, removing a tear tab, unlocking a zipping mechanism, twisting off a cap, etc.

In another embodiment, a product receptacle can include a switch, toggle, or button that can be actuated to start the charging process. In one illustrative embodiment, the switch can control the movement of a portion of the charging circuit. Prior to being activated, the portion of the charging circuit can be in an open position, such that the charging circuit is not active. When the switch is actuated, the portion of the charging circuit can move to connect the charging circuit, which proceeds to charge the charge storage device 100. In one embodiment, there can be a switch for each product or for each charge storage device 100 contained within the product receptacle. For example, a product receptacle can include a battery and ten separate products, each of which includes at least one charge storage device 100. The product receptacle can also include 10 switches corresponding to the 10 products such that each of the 10 products can be individually charged by the battery. In an alternative embodiment, a distinct battery and associated switch may be included for each of the products in the product receptacle. In another embodiment, lead wires connected to one or more batteries in the product receptacle can be manually connected to accessible electrodes that are connected to the charge storage device(s) 100 in the product receptacle. In such an embodiment, the user can manually connect the lead wires to the electrodes to begin charging a given charge storage device 100.

In one embodiment, the charge storage device 100 can be incorporated into a product, such as but not limited to, gum, cleaning sponges, snack foods, prepared foods, candies, breath strips, patches, etc. The product can be connected to the charging circuit 530 by conductive wires, conductive elastomers, conductive whiskers, conductive ink, etc. In one embodiment, the conductive wires can be connected to the first plate 110 and the second plate 130. The conductive wires can be severed from the product when the product is removed from the product receptacle 510. In another embodiment, a conductive elastomer can be used to connect the charging circuit 530 to the charge storage device 100 and to help ensure the connection's integrity. The conductive elastomer can be severed from the product when the product is removed from the product receptacle 510. In another embodiment, conductive whiskers can connect the charge storage device 100 to the charging circuit 530. In one configuration, a first conductive whisker can be connected to the charging circuit 530 and can be in electrical contact with the first plate 110. A second conductive whisker can be connected to the charging circuit 530 and can be in electrical contact with the second plate 130 such that the charging circuit 530 can charge the charge storage device 100. While the conductive whiskers are electrically connected to the charge storage device 100, they are not physically attached to the charge storage device 100. Accordingly, the product can be removed from the product receptacle without having to physically break any portion of the charging circuit 530. In yet another embodiment, conductive ink can be printed on the product receptacle 510 to connect the charge storage device 100 to the charging circuit 530. The charging circuit 530 can charge the charge storage device 100. After being charged, the charge storage device 100 can generate an electrical current when the product that includes the charge storage device 100 is used. For example, the product can be a piece of gum and the charge storage device 100 can create an electrical current in a subject's mouth when the gum is chewed.

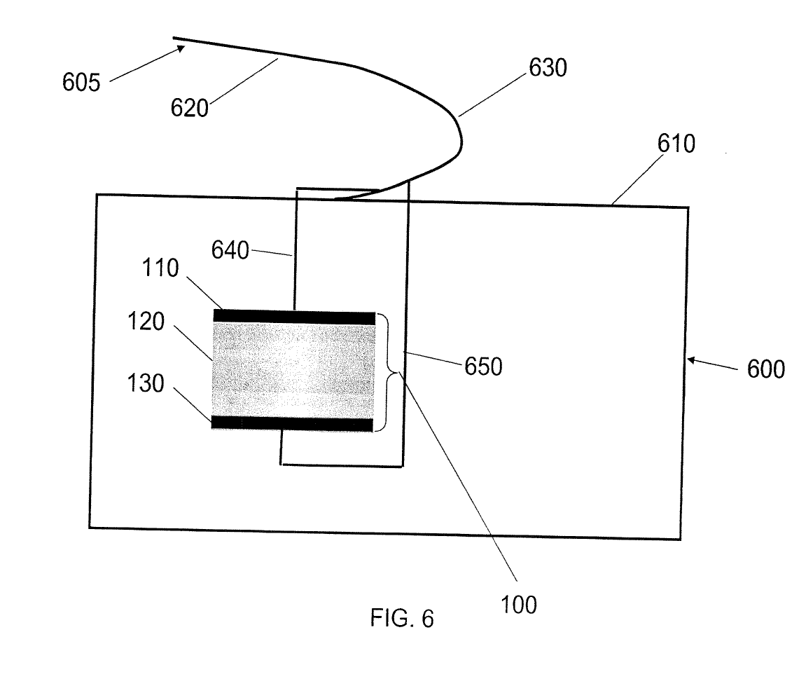

FIG. 6 illustrates a product receptacle that utilizes triboelectric charging of a charge storage device 100 in accordance with an illustrative embodiment. A product receptacle 600 includes a top portion 605 that has a first side 620 and a second side 630, and a bottom portion 610. The top portion 605 can be made of materials including, but not limited to, glass, mica, quartz, nylon, wool, silk, aluminium, etc. The bottom portion 610 can be made of materials including, but not limited to, wool, lucite, amber, wax, acrylic, polystyrene, rubber, nickel, copper, sulfur, brass, silver, gold, acetate, rayon, polyester, styrene, polyurethane, polyethylene, polypropylene, vinyl, etc. The top portion 605 can also be made of these materials if the bottom portion is made of, but not limited to, glass, mica, quartz, nylon, wool, silk, aluminium, etc. In an illustrative embodiment, opening the product receptacle 600 by at least partially separating the top portion 605 from the bottom portion 610 can create a first charge on the first side 620 of the top portion 605 and a second (opposite) charge on the second side 630 of the top portion 605. In an illustrative embodiment, prior to opening of the product receptacle 600, the top portion 605 may be adhered to the bottom portion 610 using adhesives known to those of skill in the art.

The first and second charges can be created through a triboelectric effect as known to those of skill in the art. The triboelectric effect occurs when certain materials that are in contact with one another become electrically charged when they are separated from one another. A first portion 640 of a charging circuit can electrically connect the first side 620 of the top portion 605 to the first plate 110 of the charge storage device 100. A second portion 650 of the charging circuit can electrically connect the second side 630 of the top portion 605 to the second plate 130 of the charge storage device 100. The charge storage device 100 can be connected to the top portion using conductive wires, conductive elastomers, conductive whiskers, conductive ink, etc., as described above with respect to FIG. 5. In an alternative embodiment, the charge storage device 100 may include a first electrode electrically connected to the first plate 110 and a second electrode electrically connected to the second plate 130, as in the embodiment illustrated with reference to FIG. 4. In such an embodiment, the first portion 640 of the charging circuit can electrically connect to the first electrode and the second portion 650 of the charging circuit can electrically connect to the second electrode to charge the plates of the charge storage device 100.

In an illustrative embodiment, the top portion 605 of the product receptacle 600 can be peeled back from the bottom portion 610 to generate a first charge on the first side 620 of the top portion 605, and the first side 620 can be electrically connected to the first plate 110 of the charge storage device 100. A second charge can be generated on the second side 630 of the top portion 605, and the second side 630 can be electrically connected to the second plate 130 of the charge storage device 100. Accordingly, a voltage differential based upon the first charge and the second charge can be applied across the dielectric 120, which can charge the charge storage device 100. In one embodiment, the charge storage device 100 can be incorporated into a product, such as but not limited to, gum, cleaning sponges, snack foods, prepared foods, candies, breath strips, patches etc. The product can be connected to the first portion 640 and the second portion 650 of the charging circuit by conductive wires, conductive elastomers, conductive whiskers, conductive ink, etc. The charge storage device 100 can then generate an electrical current when the product is used. For example, the product can be a cleaning sponge or pad that can generate an electrical current in an area that is cleaned with the cleaning sponge or pad.

In another embodiment, the charge storage device can be charged using piezoelectricity. A product receptacle can include a piezoelectric material such as, but not limited to, berlinite, cane sugar, quartz, Rochelle salt, topaz, tourmaline, gallium orthophosphate, langasite, etc. The piezoelectric material can be incorporated into one or more areas of the product receptacle. The areas that contain the piezoelectric material can be configured to be deformable such that the piezoelectric material can generate a voltage as it is deformed. In one embodiment, the areas that contain the piezoelectric material can be deformed by squeezing, bending, crumpling, etc. In another embodiment, a button, switch, or toggle can be used to deform the piezoelectric areas of the product receptacle. The charge storage device 100 can be attached to opposite sides of the piezoelectric material using conductive wires, conductive elastomers, conductive whiskers, conductive ink, etc., as described above with respect to FIG. 5. The voltage generated by the deforming of the areas that include piezoelectric materials can be used to charge the charge storage device 100.

In yet another embodiment, the charge storage device can be charged using magnetic induction. A product receptacle can include a coil of a conductive material. The coil can be made of any conductive material such as, but not limited to, conductive wire, conductive elastomers, conductive ink, etc. The product receptacle can also include a magnet that is configured to move rapidly through the coil. In one embodiment, the magnet is spring loaded and upon release of the spring, the spring moves the magnet through the coil. The product receptacle can include a button, switch, or toggle that can be used to initiate the movement of the magnet. In another embodiment, opening of the product receptacle can initiate the magnetic induction. The charge storage device 100 can be attached to opposite ends of the coil using conductive wires, conductive elastomers, conductive whiskers, conductive ink, etc., as described above with respect to FIG. 5. The voltage generated by moving the magnetic through the coil can be used to charge the charge storage device 100.

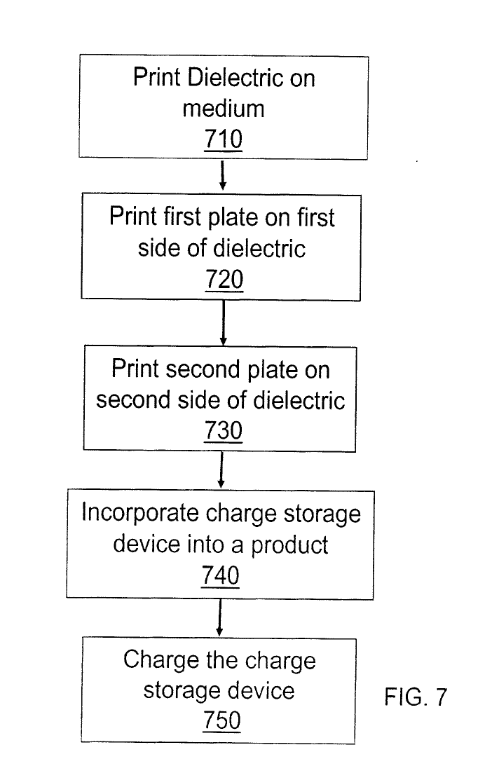

FIG. 7 is a flow diagram depicting operations performed in generating a charge storage device 100 in accordance with an illustrative embodiment. Additional, fewer, and/or different operations may be performed depending on the particular implementation. Also, one or more of the operations may be performed in a different order depending on the particular implementation. In an operation 710, a dielectric such as, but not limited to, wax can be printed on a medium. In one embodiment, the dielectric can be printed using a wax block printer. The medium can include, but is not limited to, paper such as a rice paper, dehydrated potato, etc. In an operation 720, a first plate can be printed on a first side of the dielectric. In an operation 730, a second plate can be printed on a second, opposite side of the dielectric to form the charge storage device 100. The charge storage device 100 is incorporated into a product in an operation 740. The product can be, but is not limited to, gum, a cleaning sponge, a snack food, a prepared food, a candy, a breath strip, a pill, a patch, a pad, etc. In an operation 750, the charge storage device 100 can be charged. In one embodiment, as described above, the charge storage device 100 can be charged prior to being incorporated into a product receptacle that includes the product. In other embodiments, described in detail above, the charge storage device 100 can be incorporated into a product receptacle that can charge the charge storage device 100 when the product receptacle is opened.

An inkjet printer having a resolution of 1200 dots per square inch may be used to print the first plate 110 and the second plate 130. Other resolutions may be used to print the first plate 110, the dielectric 120, and/or the second plate 130 such as, but not limited to, 600, 720, 1440, etc. dots per square inch. Once printed, the first plate 110 and the second plate 130 can be cured using ultra-violet light. In one embodiment, an inkjet printer can be used to print one or more resistors that can be electrically connected to the charge storage device 100. The uses and components of an inkjet printer are well known to those of skill in the art. In another embodiment, the dielectric 120 can be printed using, a wax printer or a phaser printer, such as a Xerox® 8550, that uses blocks of wax instead of toner to print. The uses and components of wax printing and phaser printing are well known to those of skill in the art.

The components of the charge storage device such as, but not limited to, the first plate, the second plate, the dielectric, resistors, etc., can be printed using technologies other than ink jet printers. For example, a printing press may be used to press the dielectric on the medium and can also be used to print the first and second plates on the dielectric. The processes and tools used to print using a printing press are well known to those of skill in the art.

In some embodiments, the charge storage device 100 can be encapsulated in a non-conductive coating. In one embodiment, the non-conductive coating can be waterproof. The non-conductive coating can be made of, but is not limited to, wax, sucrose, chocolate, etc. The non-conductive coated charge storage device 100 can then be incorporated into products with a high liquid content such as, but not limited to, jam, syrup, caramel, various candies, etc. The non-conductive coating acts as a barrier that separates the charge storage device 100 from moisture, and prevents the charge storage device 100 from dissipating its charge. When the non-conductive coating is broken a circuit can be formed using the charge storage device 100 as a voltage source. In one illustrative embodiment, a charge storage device 100 can be encased in wax and incorporated into a candy. When the candy is eaten, the non-conductive coating can be broken through maceration and a circuit can be formed in a subject's mouth, as described in detail above. The charge storage device 100 can generate a current in the circuit. In another embodiment, a charge storage device 100 can be incorporated into or on a pill, which can include a non-conductive coating. Digestion, chewing, or pressure can cause the non-conductive coating to break, allowing the charge storage device 100 to drive a circuit. In one embodiment, digestion of the non-conductive coating can result in an electrical current generated by the charge storage device being generated in a subject's stomach and/or other digestive organ tissue.

In another illustrative embodiment, a charge storage device 100 can be encased in a non-conductive coating by dipping the charge storage device 100 into the non-conductive coating. A non-conductive coating may also be applied using a printer. Using wax as the non-conductive coating, a phaser printer, such as a Xerox® 8550, that uses blocks of wax instead of toner to print, may be used to encase a charge storage device 100 or print a layer of wax on the charge storage device 100. The layer of wax may completely cover or encase the charge storage device 100. The uses and components of phaser printing are well known to those of skill in the art.

Numerous embodiments of the charge storage device 100 can be incorporated into various foods. In addition to producing a current, a charge storage device can increase the amount of saliva generated in the mouth of a user. Saliva can be produced by the stimulation of either or both the sympathetic nervous system and the parasympathetic nervous system. For example, stimulation of the trigeminal nerve can result in an increase in the secretion and production of saliva. While not intended to be limited by theory, electrical current generated from a charge storage device 100 may stimulate the trigeminal nerve and/or other nerves of the sympathetic and parasympathetic nervous systems. Accordingly, a charge storage device 100 can result in an increase in both saliva production and saliva secretion. In an illustrative embodiment, a charge storage device 100 can be embedded into a piece of chewing gum. Once the gum is placed in a mouth of a subject, the charge storage device 100 can increase the saliva production and saliva secretion of the user by stimulating the sympathetic nervous system and/or parasympathetic nervous system. Accordingly, a charge storage device 100 can be used to treat such conditions as Xerostomia (e.g., dry mouth) by increasing saliva production and secretion. In another embodiment, a charge storage device 100 can be incorporated into a thin film or dissolvable strip drug delivery mechanism. The current generated by the charge storage device 100 can enhance the absorption of the drug contained within the thin film drug delivery mechanism through stimulation of tissues in the mouth, stomach, and/or small intestines.

### EXAMPLES

The present compositions and methods will be understood more readily by reference to the following examples, which are provided by way of illustration and are not intended to be limiting in any way.

### Example 1

### Treatment of Dry Mouth

A charge storage device can be used as a treatment of dry mouth. The charge storage device can be embedded in a piece of chewing gum and can include electrodes that are exposed on one or more surfaces of the chewing gum. The chewing gum can be connected to a charging circuit to charge the charge storage device. Once charged, the charge storage device can be sealed in a non-conducting product receptacle. A subject can open the product receptacle and place the gum into his/her mouth. This allows the charge storage device to be administered orally to increase saliva production and secretion levels compared to the levels prior to administration of the charge storage device. The charged storage device, therefore, will be useful in treating dry mouth.

### Example 2

### Antiperspirant

One or more charge storage devices 100 can be incorporated into a patch and can be used as an antiperspirant and/or deodorant. In one embodiment, the first plate can include an aluminum complex, such us but not limited to, aluminum chloride, aluminum chlorohydrate, and aluminum-zirconium compounds. The second plate can include carbon, and the dielectric can be made of wax. The patch can include two electrodes that can be connected to the lirst plate and the second plate, respectively, and that are exposed on one surface of the patch. The patch can be incorporated into a product receptacle that includes a battery that can be connected to the first electrode and the second electrode. A non-conductive barrier can be inserted between the battery and one of the electrodes to prevent a circuit from being formed. The non-conductive barrier can be tethered to a removable portion of the product receptacle. When the removable portion is peeled from or removed from the product receptacle, the non-conductive barrier can be moved such that the battery can be electrically connected to the electrodes. Once electrically connected to the electrodes, the battery can provide a voltage differential across the first plate and the second plate to charge the charge storage device. In addition, the patch can be accessible after the peeling or removing the removable portion of the product receptacle. The patch can then be applied to a subject. The electrodes can form a circuit with a portion of the subject's skin, e.g., underarm, feet, etc. The current produced by the charge storage device through the subject's skin can aid in the iontophoretic delivery of the aluminum-based complexes. Although not intended to be limited by theory, the aluminum-based complexes may aid in the formation of plugs in sweat glands, and thus, can help prevent perspiration. The aluminum-based complexes may also interact with keratin fibrils in sweat ducts and form a physical plug that prevents sweat from reaching the surface of skin. The charge storage device results in current flowing through the underarm and results in a reduction in the amount of sweat produced by a subject compared to the amount of sweat produced prior to the application of the charge storage device.

### Example 3

### Muscle Stimulation

A patch can include one or more charge storage devices. The charge storage device(s) can be charged with a voltage source prior to being placed in a product receptacle. The patch can include electrodes that can be connected to the first and the second plate of the charge storage device and that can be located on one surface of the patch. The patch can be applied to an area of a subject's skin, e.g., under or around the eye or mouth, such that the electrodes can be in contact with the subject's skin. Electrical current can be generated by the charge storage device and the electrical current can flow across the subject's skin and cause the subject's muscles local to the placement of the electrodes to be stimulated.

### Example 4

### Breath Strips

A dissolvable strip can include a charge storage device. The dissolvable strip can include a breath enhancing flavor, such as, but not limited to, peppermint, spearmint, cinnamon, etc. The dissolvable strip can be placed in a product receptacle, which can include a removable portion. The charge storage device can include electrodes that can be connected to a first side and a second side of the removable portion, respectively. When the removable portion is peeled away from the product receptacle, a triboelectric effect causes a voltage differential across the plates of the charge storage device, which can charge the charge storage device. When the dissolvable strip is placed within a subject's mouth, the charge storage device can cause an electrical current to flow through one or more portions of the subject's mouth. The current produced from the charge storage device can act as an anti-septic and can impede the flora of the subject's mouth.

### Example 5

### Cleaning Sponge

A sponge can include a charge storage device. The charge storage device can be charged before it is placed within a product receptacle. The sponge can include a cleaning agent and/or detergent. Two electrodes can be located on one surface of the sponge and connected to the first plate and the second plate, respectively, of the charge storage device. When the sponge is placed on a conducting surface, the electrodes and the charge storage device can form a circuit through which an electrical current can flow. The electrical current generated in the circuit can be used to help loosen dirt and grease, and/or to enhance the cleaning ability of the cleaning agent and/or the detergent on the conductive surface.

One or more flow diagrams have been used herein. The use of flow diagrams is not meant to be limiting with respect to the order of operations performed. The herein-described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected” or “operably, coupled” to each other to achieve the desired functionality, and any two components capable of being so associated can also be viewed as being “operably couplable” to each other to achieve the desired functionality. Specific examples of “operably couplable” include but are not limited to physically mateablc and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances Where a convention analogous to “at least one of A, B, or C, etc,” is used, in general such a construction is intended in the sense one having skill in the art would understand the convention (e.g., “a system having at least one of A, B, or C” would include but not be limited to systems that have A alone, B alone, C alone, A and B together. A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. An apparatus comprising:
a charge storage device comprising:
a first plate configured to hold a first charge;
a second plate configured to hold a second charge, wherein the second charge is opposite of the first charge; and
a dielectric configured to separate the first plate from the second plate; and
a product receptacle configured to house the charge storage device, wherein the product receptacle is further configured to charge the first plate and the second plate upon opening of the product receptacle such that the charge storage device is able to generate an electrical current.

2. The apparatus of claim 1, wherein the charge storage device further comprises:
a first electrode mounted to the first plate; and
a second electrode mounted to the second plate.

3. The apparatus of claim 2, wherein the product receptacle comprises a battery operably connected to the first electrode and the second electrode, wherein the battery is configured to provide the first charge to the first electrode and the second charge to the second electrode upon opening of the product receptacle.

4. The apparatus of claim 1, wherein opening of the product receptacle charges the first plate with the first charge and the second plate with the second charge through triboelectric charging.

5. The apparatus of claim 1, wherein the first plate and the second plate are composed of at least one of copper, carbon, manganese dioxide, or iron.

6. (canceled)

7. The apparatus of claim 1, further comprising a food product housed in the product receptacle, wherein the food product includes the charge storage device.

8. The apparatus of claim 7, wherein the charge storage device is composed of food-grade components.

9. The apparatus of claim 7, wherein the food product comprises at least one of a gum, a candy, or a cracker.

10-12. (canceled)

13. The apparatus of claim 1, further comprising a resistor in electrical communication with at least one of the first plate and the second plate.

14. (canceled)

15. The apparatus of claim 1, wherein placement of the charge storage device in contact with a subject releases the electrical current, and wherein the electrical current is configured to increase production of saliva.

16. (canceled)

17. The apparatus of claim 1, further comprising a non-conductive layer, wherein the non-conductive layer encapsulates the charge storage device.

18-21. (canceled)

22. The apparatus of claim 1, further comprising a dissolvable strip housed in the product receptacle, wherein the charge storage device is incorporated in or on the dissolvable strip.

23. (canceled)

24. The apparatus of claim 22, wherein the electrical current is configured to reduce growth of flora within a mouth.

25. The apparatus of claim 22, wherein the electrical current is configured to combat gum disease.

26-43. (canceled)

44. A method comprising:
generating a charge storage device, wherein the generating comprises:
printing a dielectric onto a medium;
printing a first plate onto a first side of the dielectric, wherein the first plate is configured to hold a first charge;
printing a second plate onto a second side of the dielectric such that the dielectric separates the first plate from the second plate, wherein the second plate is configured to hold a second charge, and wherein the second charge is opposite of the first charge; and
incorporating the charge storage device into a product receptacle, wherein the product receptacle is configured to charge the first plate and the second plate upon opening of the product receptacle such that the charge storage device is able to generate an electrical current.

45. (canceled)

46. The method of claim 44, further comprising incorporating the charge storage device into a food product that is housed in the product receptacle.

47-52. (canceled)

53. The method of claim 44, further comprising encapsulating the charge storage device with a non-conductive layer.

54. (canceled)

55. (canceled)

56. The method of claim 44, further comprising printing a resistor so that the resistor is electrically connected to at least one of the first plate or the second plate.

57. (canceled)

58. The method of claim 44, further comprising incorporating a battery into the product receptacle.

59. The method of claim 58, wherein the battery is configured to provide the first charge to the first plate and the second charge to the second plate upon opening of the product receptacle.

60. The method of claim 44, further comprising, upon opening of the product receptacle, providing the first charge to the first plate and the second charge to the second plate via triboelectric charging.

61. The method of claim 44, wherein the electrical current increases production of saliva.

62-65. (canceled)

66. The method of claim 44, further comprising incorporating the charge storage device in or on a dissolvable strip that is housed in the product receptacle.

67-71. (canceled)

72. An apparatus comprising:
a charge storage device comprising:
a first plate configured to hold a first charge;
a second plate configured to hold a second charge, wherein the second charge is opposite of the first charge; and
a dielectric configured to separate the first plate from the second plate; and
an edible product configured to house the charge storage device.

73. The apparatus of claim 72, further comprising a product receptacle configured to house the edible product.

74. The apparatus of claim 73, wherein the product receptacle is configured to charge the first plate and the second plate upon opening of the product receptacle such that the charge storage device is able to generate an electrical current.

75. The apparatus of claim 73, wherein the first charge is applied to the first plate and the second charge is applied to the second plate prior to placement of the edible product into the product receptacle.

76. A method comprising:
generating a charge storage device, wherein the generating comprises:
printing a dielectric onto a medium;
printing a first plate onto a first side of the dielectric, wherein the first plate is configured to hold a first charge;
printing a second plate onto a second side of the dielectric such that the dielectric separates the first plate from the second plate, wherein the second plate is configured to hold a second charge, and wherein the second charge is opposite of the first charge; and
incorporating the charge storage device into an edible product.
